(19) United States
(12) Patent Application Publication
(10) Pub. No.: US 2018/0303416 A1
(43) Pub. Date: Oct. 25, 2018

(54) WRIST-WORN TYPE ELECTRONIC DEVICE

(71) Applicant: VitalSigns Technology Co., Ltd., Hsinchu County (TW)

(72) Inventor: Jih-Liang JUANG, Hsinchu County (TW)

(21) Appl. No.: 15/657,225

(22) Filed: Jul. 24, 2017

(30) Foreign Application Priority Data
Apr. 24, 2017 (CN) 201710269877.4

(51) Int. Cl. A61B 5/00; H01L 31/16; A61B 5/0408; A61B 5/0205; G04G 21/02; G04B 37/00

(52) U.S. Cl. CPC A61B 5/681; H01L 31/16; A61B 5/04085; A61B 5/021; A61B 5/0205; G04G 21/025; G04B 37/0008; A61B 5/0006

(57) ABSTRACT

A wrist-worn type electronic device includes a wrist-type electronic device includes a device body, an outer frame, a physiological information induction module and a control unit. The device body includes a front surface and a back surface which are opposite each other. The outer frame surrounds the front surface. The physiological information induction module includes a pair of electrocardiogram electrodes which are arranged opposite on the device body, and one of the electrocardiogram electrodes is disposed on the outer frame. The control unit is electrically connected these electrocardiographic electrodes.

### RELATED APPLICATIONS

This application claims priority to China Application Serial Number 201710269877.4, filed Apr. 24, 2017, which is herein incorporated by reference.

### BACKGROUND

### Field of Disclosure

The present disclosure relates to a wrist-worn type electronic device. More particularly, the present disclosure relates to a wrist-worn type electronic device for sensing physiological signals.

### Description of Related Art

In order to meet market trends and consumer expectations, as the advancement of electronic and wireless communications technology continues, a wrist-worn type electronic device (such as a smart watch, etc.) is able to provide more information services through to the support of a smart phone. For example, the wrist-worn type electronic devices are able to read basic personal physiological parameters (e.g., blood pressure, pulse, and body temperature) in the daily life of a wearer.

However, types of basic personal physiological parameters able to be read by the conventional wrist-worn type electronic device are still few, and the conventional wrist-worn type electronic device is needed to be preserved additional configuration spaces therein for placing individual sensor heads of different basic personal physiological parameters mentioned above, thereby increasing the complexity of component configuration on the conventional wrist-worn type electronic device. Therefore, how to provide a solution to effectively overcome the aforementioned inconvenience and shortages is a serious concern for many industries.

### SUMMARY

An aspect of the disclosure is to provide a wrist-worn type electronic device to resolve the aforementioned inconvenience and deficiencies.

According to one embodiment, a wrist-worn type electronic device includes a device body, a signal indicating module, an outer frame, a physiological information induction module, a wireless communication module and a control unit. The device body includes a front surface and a back surface which are opposite to each other. The signal indicating module is disposed on the device body. The outer frame surrounds the signal indicating module. The physiological information induction module includes a first electrocardiogram electrode and a second electrocardiogram electrode which are oppositely arranged on the device body. The first electrocardiogram electrode is disposed on the outer frame. The wireless communication module is disposed in the device body, for wirelessly and electrically connected to an external device. The control unit is electrically connected to the first electrocardiogram electrode, the second electrocardiogram electrode, the wireless communication module and the signal indicating module.

Thus, according to the aforementioned configuration of the embodiment, as the wrist-worn type electronic device integrates the electrocardiogram electrode into the outer frame, the configuration space of the wrist-worn type electronic device can be saved so as to decrease the complexity of component configuration on the wrist-worn type electronic device. In addition, the wrist-worn type electronic device of the embodiment also provides more types of basic personal physiological parameters.

In one or more embodiments of the disclosure, the outer frame includes an annular conductive frame. The entire annular conductive frame is the first electrocardiogram electrode.

In one or more embodiments of the disclosure, the first electrocardiogram electrode includes a plurality of conductors spaced to be arranged on the outer frame.

In one or more embodiments of the disclosure, the physiological information induction module further includes an optical transceiver set. The optical transceiver set is disposed on the device body for emitting light to a human skin and receiving reflective light from the human skin.

In one or more embodiments of the disclosure, the second electrocardiogram electrode and the optical transceiver set are both disposed on the back surface of the device body, and the second electrocardiogram electrode surrounds the optical transceiver set.

In one or more embodiments of the disclosure, the device body includes a first recessed portion, a second recessed portion, a light shielding portion, a first light transmissive sheet and a second light transmissive sheet. The first recessed portion and the second recessed portion are formed on the back surface of the device body, and the light shielding portion is arranged between the first recessed portion and the second recessed portion. The first light transmissive sheet is totally received within the first recessed portion, and the second light transmissive sheet is totally received within the second recessed portion. The optical transceiver set includes a photodiode unit and a first light emitting diode unit. The first light emitting diode unit is disposed in the first recessed portion for emitting lights to the human skin through the first light transmissive sheet. The photodiode unit is disposed in the second recessed portion for receiving reflective lights from the human skin through the second light transmissive sheet.

In one or more embodiments of the disclosure, the optical transceiver set further includes a second light emitting diode unit. The second light emitting diode unit is disposed in the first recessed portion for emitting light to the human skin through the first light transmissive sheet, and the first recessed portion surrounds the second recessed portion.

In one or more embodiments of the disclosure, the signal indicating module includes a watch face image, a plurality of indicating pointers and a plurality of driving motors. The watch face image is disposed on one surface of the device body. The indicating pointers are rotatably disposed on the watch face image, and the indicating pointers indicate at least one detail of physiological information. The driving motors are disposed in the device body, and electrically connected to the control unit. Each of the driving motors individually connects to control one of the indicating pointers.

In one or more embodiments of the disclosure, the wrist-worn type electronic device further includes a manual switch. The manual switch is disposed on the device body, electrically connected to the control unit, for being manually operated to enable and disable the wireless communication module to electrically connect to the external device.

According to another embodiment, a wrist-worn type electronic device includes a device body, a physiological information induction module, a signal indicating module, a wireless communication module and a control unit. The device body includes a first recessed portion, a second recessed portion, a light shielding portion, a first light transmissive sheet and a second light transmissive sheet. The light shielding portion is arranged between the first recessed portion and the second recessed portion, the first light transmissive sheet is totally received within the first recessed portion, and the second light transmissive sheet is totally received within the second recessed portion. The physiological information induction module includes a circuit board, a first light emitting diode unit and a second light emitting diode unit. The circuit board is disposed in the device body. The first light emitting diode unit is mounted on the circuit board, and disposed in the first recessed portion, for emitting light to the human skin through the first light transmissive sheet. The photodiode unit is mounted on the circuit board, and disposed in the first recessed portion for receiving reflective lights from the human skin through the second light transmissive sheet. The signal indicating module is disposed on the device body. The wireless communication module is disposed in the device body, for wirelessly and electrically connected to an external device. The control unit is electrically connected to the photodiode unit, the first light emitting diode unit, the wireless communication module and the signal indicating module.

In one or more embodiments of the disclosure, the physiological information induction module further includes a second light emitting diode unit. The second light emitting diode unit is disposed in the first recessed portion, electrically connected to the control unit. The second recessed portion surrounds the first recessed portion.

In one or more embodiments of the disclosure, the physiological information induction module further includes a first electrocardiogram electrode and a second electrocardiogram electrode which are oppositely arranged on the device body, and both electrically connected to the control unit. The first electrocardiogram electrode is an annular conductive frame surrounding the signal indicating module.

In one or more embodiments of the disclosure, the signal indicating module includes a watch face image, a plurality of indicating pointers and a plurality of driving motors. The watch face image is disposed on one surface of the device body. The indicating pointers are rotatably disposed on the watch face image, and the indicating pointers indicate at least one physiological information. The driving motors are disposed in the device body, and electrically connected to the control unit. Each of the driving motors individually connects to control one of the indicating pointers.

In one or more embodiments of the disclosure, the wrist-worn type electronic device further includes a manual switch. The manual switch is disposed on the device body, electrically connected to the control unit, for being manually operated to enable and disable the wireless communication module to electrically connect to the external device.

The above description is merely used for illustrating the problems to be resolved, the technical methods for resolving the problems and their efficacies, etc. The specific details of the present disclosure will be explained in the embodiments below and related drawings.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure. In the drawings,

FIG. 1 is a front view of a wrist-worn type electronic device according to one embodiment of the disclosure;

FIG. 2 is an electrical block diagram of the wrist-worn type electronic device of FIG. 1;

FIG. 3 is a rear view of a wrist-worn type electronic device according to one embodiment of the disclosure;

FIG. 4 is a partial cross-sectional view of FIG. 3 taken along A-A;

FIG. 5 is a front view of a wrist-worn type electronic device according to one embodiment of the disclosure;

FIG. 6 is a partial cross-sectional view of FIG. 3 taken along B-B;

FIG. 7 is a partial cross-sectional view and a schematic operational view of a wrist-worn type electronic device according to one embodiment of the disclosure in which the sectional position is the same as that in FIG. 6;

FIG. 8 is a rear view of a wrist-worn type electronic device according to one embodiment of the disclosure;

FIG. 9A and FIG. 9B are schematic operational views of a wrist-worn type electronic device according to one embodiment of the disclosure; and

FIG. 10 is an electrical block diagram of the wrist-worn type electronic device of FIG. 1.

### DESCRIPTION OF THE EMBODIMENTS

Reference will now be made in detail to the present embodiments of the disclosure, examples of which are illustrated in the accompanying drawings. Wherever possible, the same reference numbers are used in the drawings and the description to refer to the same or like parts. According to the embodiments, it will be apparent to those skilled in the art that various modifications and variations can be made to the structure of the present disclosure without departing from the scope or spirit of the disclosure.

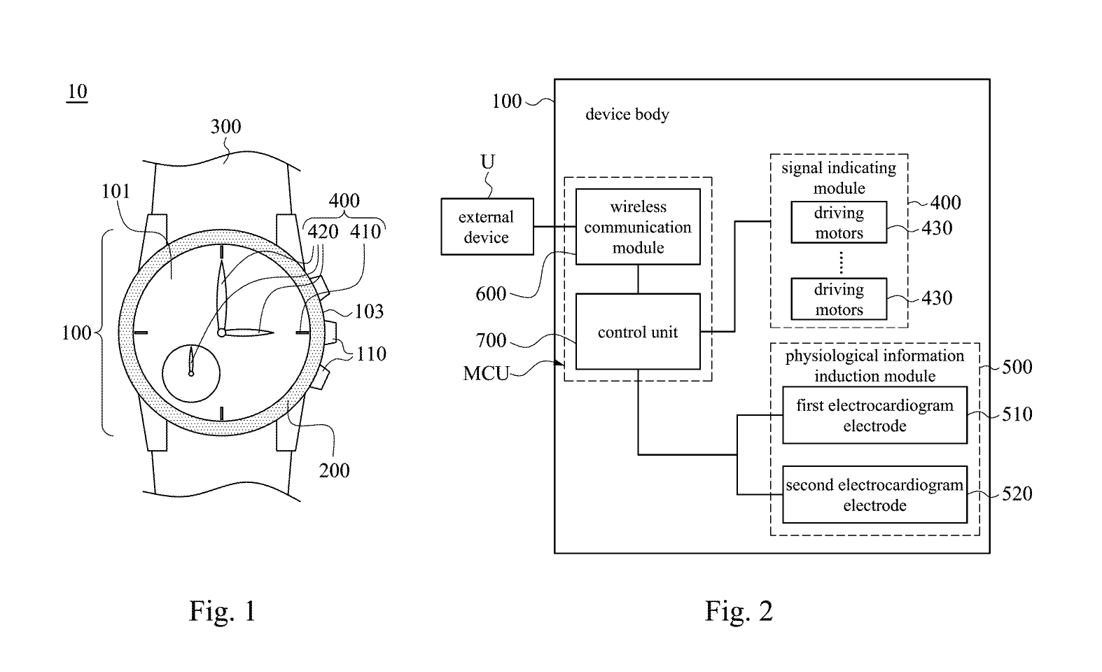

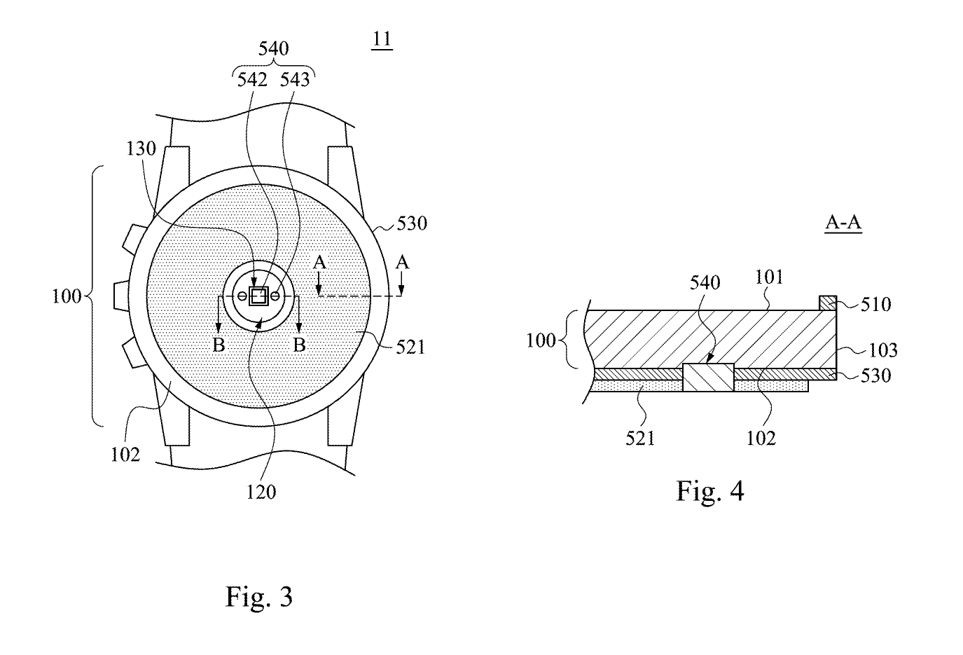

Reference is now made to FIG. 1 and FIG. 2, in which FIG. 1 is a front view of a wrist-worn type electronic device 10 according to one embodiment of the disclosure, and FIG. 2 is an electrical block diagram of the wrist-worn type electronic device 10 of FIG. 1. As shown in FIG. 1 and FIG. 2, the wrist-worn type electronic device 10 includes a device body 100, a wearing element 300, a signal indicating module 400, a physiological information induction module 500, a wireless communication module 600 and a control unit 700. The wearing element 300 is connected to the device body 100, and is used to be worn on a wearer's wrist. The device body includes a front surface 101, a back surface 102 (FIG. 3) and side surfaces 103. The back surface 102 is facing to the wearer's wrist, the front surface 101 is opposite to the back surface 102, and each of the side surfaces 103 is disposed between the front surface 101 and the back surface 102. The signal indicating module 400 is disposed on the device body 100. The wrist-worn type electronic device 10 further includes an outer frame 200 disposed on the device body 100. For example, the outer frame 200 adjoins and surrounds the front surface 101, and surrounds the signal indicating module 400.

The physiological information induction module 500 includes a first electrocardiogram (ECG) electrode 510 and a second electrocardiogram (ECG) electrode 520. The first electrocardiogram (ECG) electrode 510 and the second electrocardiogram (ECG) electrode 520 are oppositely arranged on the device body 100 for respectively contacting different types of human skin in which the first electrocardiogram (ECG) electrode 510 and the second electrocardiogram (ECG) electrode 520 collectively generate electrical pulses by sensing the heartbeats of a wearer's heart through the different human skins respectively, and translate the electrical pulses into electrocardiogram signals. In this embodiment, the first electrocardiogram electrode 510 is disposed on the outer frame 200 of the device body 100. For example, the outer frame 200 includes an annular conductive frame and connecting parts (not shown in figures), and the entire part of the annular conductive frame can be used as the first electrocardiogram electrode 510, and the first electrocardiogram electrode 510 is electrically connected to the control unit 700 through one of the connecting parts. The first electrocardiogram electrode 510 is monolithically formed on the outer frame 200. In the embodiment, the second electrocardiogram (ECG) electrode 520 is not limited to be configured on any location of the device body 100 as long as the second electrocardiogram (ECG) electrode 520 is able to contact human skin. The wireless communication module 600 is disposed in the device body 100, for wirelessly and electrically connected to an external device U. The external device U for example cam be a smartphone, a user computer (NB or PC), or a cloud device. The control unit 700 is electrically connected to the first electrocardiogram (ECG) electrode 510, the second electrocardiogram (ECG) electrode 520, the wireless communication module 600 and the signal indicating module 400, and the control unit 700 is used to send the electrocardiogram signals provided by the first electrocardiogram (ECG) electrode 510, the second electrocardiogram (ECG) electrode 520 to the external device U with the wireless communication module 600, to receive feedback data from the external device U to report at least one detail of physiological information on the signal indicating module 400. In this embodiment, the wireless communication module 600 and control unit 700 can be integrated as one in a micro controller chip (MCU), however, the disclosure is not limited thereto.

FIG. 3 is a rear view of a wrist-worn type electronic device 11 according to one embodiment of the disclosure, and FIG. 4 is a partial cross-sectional view of FIG. 3 taken along A-A. As shown in FIG. 3 and FIG. 4, the wrist-worn type electronic device 11 of FIG. 3 is substantially the same as the wrist-worn type electronic device 10 of FIG. 1 (the same elements are respectively indicated with the same reference numbers), except that the second electrocardiogram (ECG) electrode 521 in the wrist-worn type electronic device 11 of FIG. 3 is disposed on the back surface 102 of the device body 100 for directly contacting with the human skin of the wrist. Specifically, the device body 100 is further provided with an insulated outer layer 530. The insulated outer layer 530 is disposed on the back surface 102 of the device body 100 between the first electrocardiogram (ECG) electrode 510 and the second electrocardiogram (ECG) electrode 521, and the insulated outer layer 530 isolates the second electrocardiogram (ECG) electrode 521 and device body 100, or the second electrocardiogram (ECG) electrode 521 and the first electrocardiogram (ECG) electrode 510 for avoiding signal noise caused by the sweat of the hands.

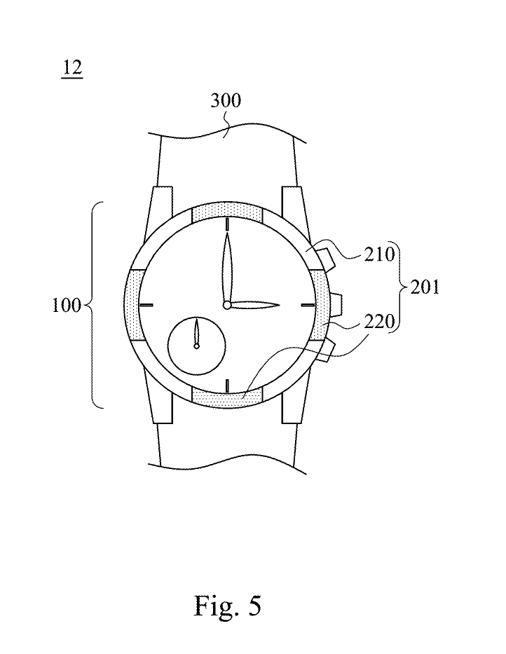

FIG. 5 is a front view of a wrist-worn type electronic device 12 according to one embodiment of the disclosure. As shown in FIG. 5, the wrist-worn type electronic device 12 of FIG. 5 is substantially the same as the wrist-worn type electronic device 10 of FIG. 1, (the same elements are respectively indicated with the same reference numbers) except that the outer frame 201 in the wrist-worn type electronic device 12 of FIG. 5 includes an annular insulation body 210 and a plurality of conductors 220 spaced to be arranged on the annular insulation body 210. For example, the conductors 220 are equidistantly arranged on the annular insulation body 210 so as to facilitate the decoration and styling of the outer frame 201. All of the conductors 220 are collectively electrically connected to the control unit 700 (FIG. 2), thus, all of the conductors 220 are collectively referred to as the above-mentioned first electrocardiographic electrode.

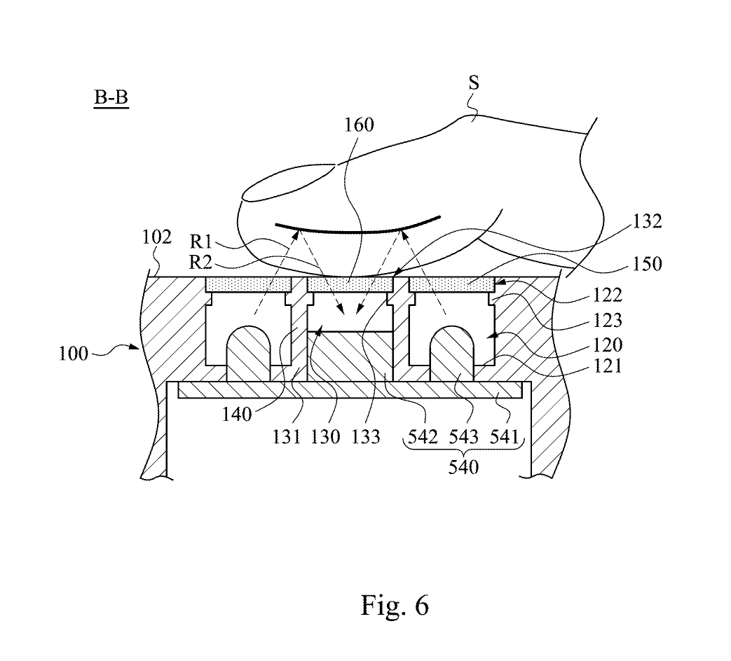

FIG. 6 is a partial cross-sectional view of FIG. 3 taken along B-B. As shown in FIG. 3 and FIG. 6, at least one difference between the wrist-worn type electronic device 11 of FIG. 3 and the wrist-type electronic device 10 of FIG. 1 is that the physiological information induction module 500 further includes an optical transceiver set 540. The optical transceiver set 540 is disposed on the device body 100 for emitting lights R1 to a human skin S and receiving reflective lights R2 from the human skin S.

Thus, as shown in FIG. 2 and FIG. 6, after the control unit 700 converts the lights R1 into specific signals and sends the specific signals to the external device U, by calculation and analysis of the external device U, the control unit 700 is allow to send processed feedback information to the signal indicating module 400 for indicating the physiological information of heartbeats and blood vessel blood volume (i.e., blood pressure). In the embodiment, the optical transceiver set 540 is disposed on the back surface 102 of the device body 100. Specifically, the second electrocardiogram electrode 521 and the optical transceiver set 540 are both disposed on the back surface 102 of the device body 100, and the second electrocardiogram electrode 521 surrounds the optical transceiver set 540.

Furthermore, as shown in FIG. 3 and FIG. 6, the device body 100 includes a first recessed portion 120, a second recessed portion 130 and a light shielding portion 140. The first recessed portion 120 and the second recessed portion 130 are both formed on the back surface 102 of the device body 100, and the light shielding portion 140 is arranged between the first recessed portion 120 and the second recessed portion 130. Specifically, the light shielding portion 140 is presented as a surrounding wall surrounding to form the second recessed portion 130 and surrounded by the first recessed portion 120. Also, the first recessed portion 120 is provided with a first bottom portion 121 and a first opening 122 which are oppositely formed on two ends of the first recessed portion 120, and the first opening 122 is connected to the back surface 102 of the device body 100. The second recessed portion 130 is provided with a second bottom portion 131 and a second opening 132 which are oppositely formed on two ends of the second recessed portion 130, and the second opening 132 is connected to the back surface 102 of the device body 100. The device body 100 is further provided with a first position-limited flange 123 and a second position-limited flange 133. The first position-limited flange 123 is protrusively formed on an inner surface of the first recessed portion 120. The second position-limited flange 133 is protrusively formed on an inner surface of the second recessed portion 130. The device body 100 is further provided with a first light transmissive sheet 150 and a second light transmissive sheet 160. The first light transmissive sheet 150 is totally received within the first recessed portion 120, for example, the first light transmissive sheet 150 is flat disposed on the first position-limited flange 123 so that the first light transmissive sheet 150 can be fixedly held in the first opening 122 of the first recessed portion 120 without extending outwards from the space of the first recessed portion 120.

The second light transmissive sheet 160 is totally received within the second recessed portion 130, for example, the second light transmissive sheet 160 is flat disposed on the second position-limited flange 133 so that the second light transmissive sheet 160 can be fixedly held in the second opening 132 of the second recessed portion 130 without extending outwards from the space of the second recessed portion 130. The first light transmissive sheet 150 and the second light transmissive sheet 160 respectively include general light-penetrating material, however, the disclosure is not limited thereto. In another embodiment, each of the first light transmissive sheet 150 and the second light transmissive sheet 160 also can a focus lens for focusing light.

The optical transceiver set 540 includes a circuit board 541, a photodiode unit 542 and two first light emitting diode unit 543. The circuit board 541 is disposed in the device body 100. The first light emitting diode units are respectively soldered to be mounted on the circuit board 541, and the first light emitting diode units are disposed in the first recessed portion 120 for emitting lights R1 to the human skin R through the first light transmissive sheet 150. The photodiode unit 542 is soldered to be mounted on the circuit board 541 between the two first light emitting diode units 543, and is received within the second recessed portion 130 for receiving reflective lights R2 from the human skin S through the second light transmissive sheet 160. It is noted, the first light emitting diode units 543 respectively are not limited in colors and wavelengths. The first light emitting diode units 543 for example can be a green light emitting diode, a red light emitting diode, or an infrared light emitting diode, however, the disclosure is not limited thereto.

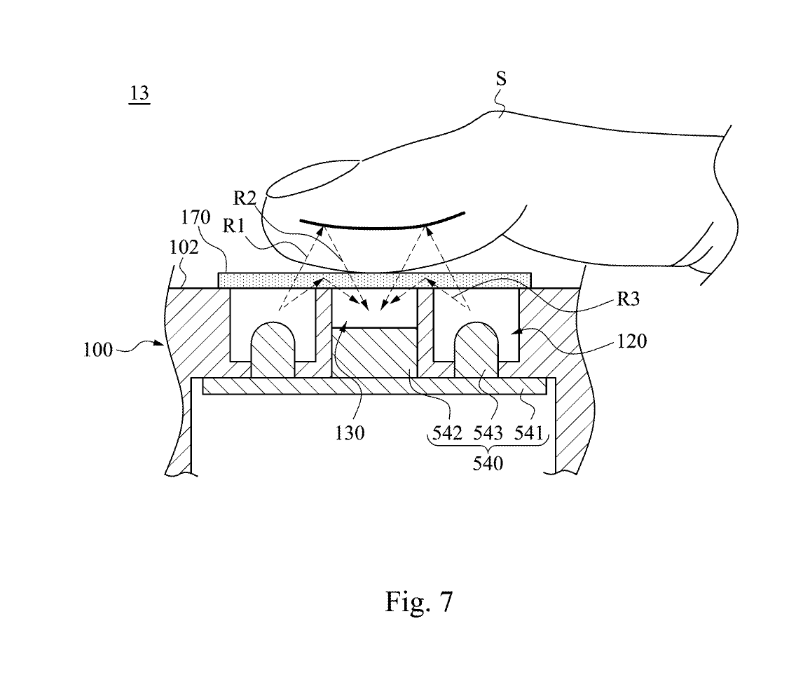

FIG. 7 is a partial cross-sectional view and a schematic operational view of a wrist-worn type electronic device 13 according to one embodiment of the disclosure in which the sectional position is the same as that in FIG. 6. As shown in FIG. 7, the wrist-worn type electronic device 13 of FIG. 7 is substantially the same as the wrist-worn type electronic device 11 of FIG. 6, (the same elements are respectively indicated with the same reference numbers) except that the device body 100 in the wrist-worn type electronic device 13 of FIG. 7 is only provided with one third light transmissive sheet 170 rather than the first light transmissive sheet 150 and the second light transmissive sheet 160. The third light transmissive sheet 170 covers the back surface 102, the first recessed portion 120 and the second recessed portion 130 at the same time. Since the wrist-worn type electronic device 13 of FIG. 7 is only provided with the single one third light transmissive sheet 170, cost and procedure steps can be saved.

Thus, since the third light transmissive sheet 170 in FIG. 7 covers the back surface 102, the first recessed portion 120 and the second recessed portion 130 at the same time, a part of lights R3 not penetrated through the third light transmissive sheet 170 from the first light emitting diode units 543 might be reversely guided to the photodiode unit 542 by the third light transmissive sheet 170 directly, thereby generating a great noise so as to reduce the accuracy of the physiological information read by the photodiode unit 542. However, in FIG. 6, since the first light transmissive sheet 150 is totally received within the first recessed portion 120, and the second light transmissive sheet 160 is totally received within the second recessed portion 130, the part of lights not penetrated through the first light transmissive sheet 150 from the first light emitting diode units 543 will not be reversely guided to the photodiode unit 542 by the first light transmissive sheet 150 so as to avoid reducing the accuracy of the physiological information read by the photodiode unit 542.

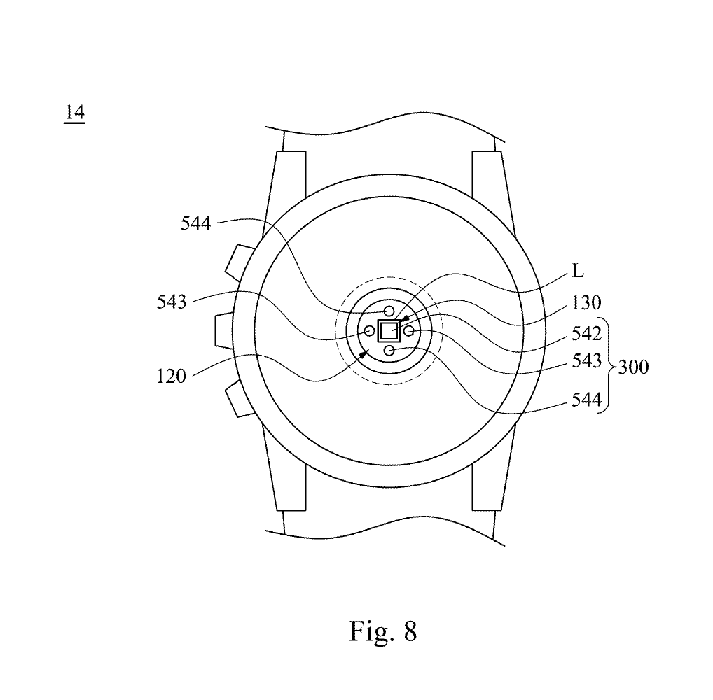

FIG. 8 is a rear view of a wrist-worn type electronic device 14 according to one embodiment of the disclosure. As shown in FIG. 8, the wrist-worn type electronic device 14 of FIG. 8 is substantially the same as the wrist-worn type electronic device 11 of FIG. 6, and the same elements are respectively indicated with the same reference numbers. At least one difference between the wrist-worn type electronic device 14 of FIG. 8 and the wrist-worn type electronic device 11 of FIG. 6 is that the optical transceiver set 540A in the wrist-worn type electronic device 14 of FIG. 8 further includes two second light emitting diode units 544. The second light emitting diode units 544 are both disposed in the first recessed portion 120 for emitting lights to the human skin through the first light transmissive sheet 150.

For example, in the embodiment, the first light emitting diode units 543 and the second light emitting diode units 544 collectively surround the photodiode unit 542, and the first light emitting diode units 543 and the second light emitting diode units 544 are arranged alternately. Also, when the second recessed portion 130 is approximately shaped as a rectangle, the first light emitting diode units 543 and the second light emitting diode units 544 respectively correspond to the four lateral sides L of the second recessed portion 130.

It is noted, each of the second light emitting diode units 544 and each of the first light emitting diode units 543 is different in type. The second light emitting diode units 544 respectively are not limited in colors and wavelengths. The second light emitting diode units 544 for example can be a green light emitting diode, a red light emitting diode, or an infrared light emitting diode, however, the disclosure is not limited thereto. One of the first light emitting diode unit 543 and the second light emitting diode unit 544 is an infrared light emitting diode.

Back to FIG. 1 and FIG. 2, for example, when the wrist-worn type electronic device 10 is a pointer watch, the device body 100 can be a caliber of the pointer watch, the signal indicating module 400 can be indicating pointers of the pointer watch, the outer frame 200 can be a bezel of the pointer watch surrounding the watch face thereof, and the wearing element 300 can be two watch bands respectively coupled to two opposite sides of the device body 100.

In great details, the signal indicating module 400 includes a watch face image 410, a plurality of indicating pointers 420 (e.g., an hour hand, a minute hand and a second hand) and a plurality of driving motors 430. The watch face image 410 is disposed on the front surface 101 of the device body 100. The indicating pointers 420 are rotatably disposed on the watch face image 410. The driving motors 430 are disposed in the device body 100, and electrically connected to the control unit 700. Each of the driving motors 430 individually connects to control one of the indicating pointers 420 so that the control unit 700 is allowed to control the indicating pointers 420 to indicate physiological information and time information. For example, but not to be a limitation in the disclosure, in the embodiment, the wrist-worn type electronic device 10 uses the minute hand to indicate systolic blood pressure of blood pressure, uses the hour hand to indicate diastolic blood pressure of blood pressure, and uses the second hand to indicate heartbeats. Also, in another embodiment, the wrist-worn type electronic device 10 uses the indicating pointers 420 to indicate whether the blood pressure and the heartbeat are normal, or to indicate the degrees (i.e., high, normal or low) of the blood pressure and the heartbeat.

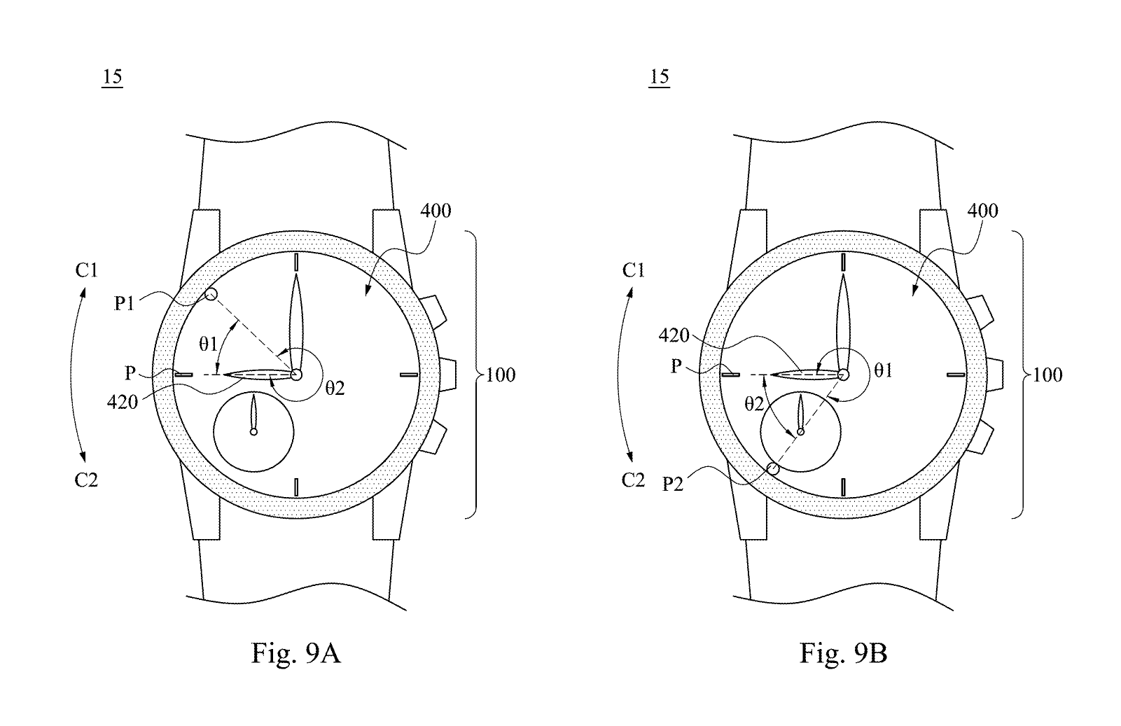

FIG. 9A and FIG. 9B are schematic operational views of a wrist-worn type electronic device 15 according to one embodiment of the disclosure. As shown in FIG. 2 and FIG. 9A, when the wrist-worn type electronic device 15 is instructed for indicating any information through at least one of the indicating pointers 420, the control unit 700 respectively obtain a first rotation angle θ1 and a second rotation angle θ2 in accordance with a current position P of one of the pointer 420 and the information representative position (hereinafter referred to as a target position P1) in which the first rotation angle θ1 is an angle which one of the indicating pointers 420 is going to rotate to the target position P1 from the current position P in the clockwise direction C1, and the second rotation angle θ2 is another angle which the same indicating pointer 420 is going to rotate to the target position P1 from the current position P in the counterclockwise direction C2. Next, the control unit 700 determines that whether the first rotation angle θ1 is not greater than the second rotation angle θ2. If yes, the control unit 700 controls the indicating pointer 420 to rotate the first rotation angle θ1 to the target position P1 from the current position P in the clockwise direction C1; otherwise, the control unit 700 controls the indicating pointer 420 to rotate the second rotation angle θ2 to the target position P1 from the current position P in the counterclockwise direction C2. Therefore, the rotation time of the indicating pointer 420 and power consumption can be reduced.

For example, as shown in FIG. 2 and FIG. 9A, when the wrist-worn type electronic device 15 is instructed to rotate the indicating pointer 420 which is originally pointed to a position of 9 o'clock (i.e., the current position P) of the pointer watch to an target position P1, the control unit 700 calculates to obtain the first rotation angle θ1 is 45°, and the second rotation angle θ2 is 315°. Next, since the control unit 700 determines that the first rotation angle θ1 is not greater than the second rotation angle θ2, in response to the determination of the first rotation angle θ1 not greater than the second rotation angle θ2, the control unit 700 controls the indicating pointer 420 rotating 45° to arrive the target position P1 from the current position P in the clockwise direction C1.

On the other hand, as shown in FIG. 2 and FIG. 9B, when the wrist-worn type electronic device 15 is instructed to rotate the indicating pointer 420 which is originally pointed to the position of 9 o'clock (i.e., the current position P) of the pointer watch to another target position P2, the control unit 700 calculates to obtain the first rotation angle θ1 is 315°, and the second rotation angle θ2 is 45°. Next, since the control unit 700 determines that the first rotation angle θ1 is greater than the second rotation angle θ2, in response to the determination of the first rotation angle θ1 being greater than the second rotation angle θ2, the control unit 700 controls the indicating pointer 420 rotating 45° to arrive the another target position P2 from the current position P in the counterclockwise direction C2. However, the disclosure is not limited thereto, in another embodiment, the wrist-worn type electronic device is adoptable to a digital watch or an electronic wristband. In another embodiment, when the wrist-worn type electronic device (not shown) is a digital watch, the signal indicating module 400 is a display screen. The display screen is located on the front side of the device body and is electrically connected to the control unit 700 so that the control unit 700 controls the display screen to display physiological information and time information.

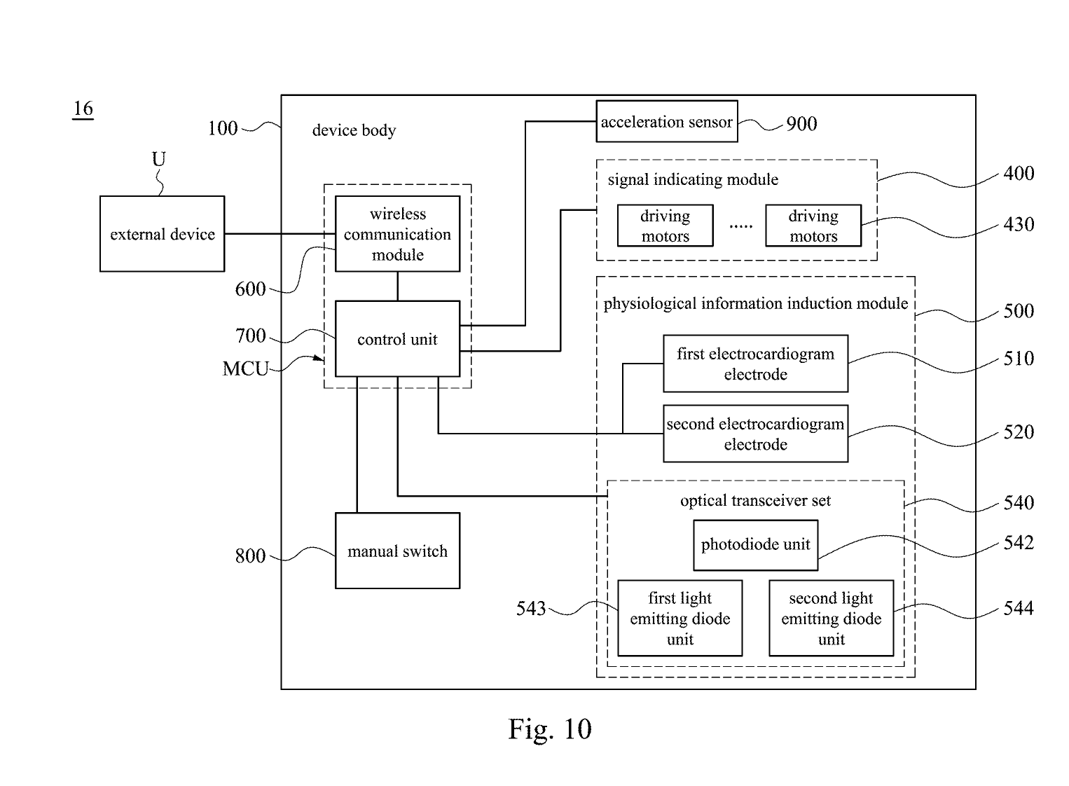

In this embodiment, as shown in FIG. 10, the wrist-worn type electronic device 16 further includes a manual switch 800 disposed on the device body 100, electrically connected to the control unit 700, for being manually operated to enable and disable the wireless communication module 600 to electrically connect to the external device U. Since the power consumption of a digital watch capable of wirelessly connecting to the external device U is greater than the power consumption of a normal pointer watch, it cannot be satisfied for a wearer wearing the digital watch for a long time. Thus, when not exchanging information with the external device U, a user can manually turn off the wireless communication module 600 of the wrist-worn type electronic device 16 by the manual switch 800, the power consumption of the wrist-worn type electronic device 16 can be greatly reduced so as to be advantageous for a wearer wearing the wrist-worn type electronic device 16 for a long time.

Specifically, as shown in FIG. 1 and FIG. 10, the manual switch 800 for example, is provided with one or multiple pressing buttons 110. The pressing buttons 110 are configured on one of the side surfaces 103. Through the operation of long pressing, single-time short pressing or multiple short pressing of the pressing buttons 110, the wrist-worn type electronic device 16 not only can adjust the time and check the State of charge (SOC) of battery, but also enable or disable the wireless communication module 600 to be wirelessly connected to the external device U.

Also, as shown in FIG. 10, the wrist-worn type electronic device 16 further includes an acceleration sensor 900 disposed in the device body 100, electrically connected to the control unit 700 for counting daily stepping, sensing sleeping and elimination of motion noises in simultaneous measurement of the electrocardiogram signals and heartbeats described above. The acceleration sensor 900 may also be referred to as a Gravity-sensor, which is able to sense the acceleration of a three-dimensional space.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims and their equivalents.

What is claimed is:

1. A wrist-worn type electronic device, comprising:
a device body comprising a front surface and a back surface which are opposite to each other;
a signal indicating module disposed on the device body;
an outer frame surrounding the signal indicating module;
a physiological information induction module comprising a first electrocardiogram electrode and a second electrocardiogram electrode which are oppositely arranged on the device body, wherein the first electrocardiogram electrode is disposed on the outer frame;
a wireless communication module disposed in the device body, for wirelessly and electrically connected to an external device; and
a control unit electrically connected to the first electrocardiogram electrode, the second electrocardiogram electrode, the wireless communication module and the signal indicating module.

2. The wrist-worn type electronic device of claim 1, wherein the outer frame comprises an annular conductive frame, and the entire annular conductive frame is the first electrocardiogram electrode.

3. The wrist-worn type electronic device of claim 1, wherein the first electrocardiogram electrode comprises a plurality of conductors spaced to be arranged on the outer frame.

4. The wrist-worn type electronic device of claim 1, wherein the physiological information induction module further comprises an optical transceiver set, the optical transceiver set is disposed on the device body for emitting light to a human skin and receiving reflective light from the human skin.

5. The wrist-worn type electronic device claim 4, wherein the second electrocardiogram electrode and the optical transceiver set are both disposed on the back surface of the device body, and the second electrocardiogram electrode surrounds the optical transceiver set.

6. The wrist-worn type electronic device claim 4, wherein the device body comprises a first recessed portion, a second recessed portion, a light shielding portion, a first light transmissive sheet and a second light transmissive sheet, the first recessed portion and the second recessed portion are formed on the back surface of the device body, and the light shielding portion is arranged between the first recessed portion and the second recessed portion, the first light transmissive sheet is totally received within the first recessed portion, and the second light transmissive sheet is totally received within the second recessed portion; and
the optical transceiver set comprises a photodiode unit and a first light emitting diode unit, the first light emitting diode unit is disposed in the first recessed portion for emitting light to the human skin through the first light transmissive sheet, and the photodiode unit is disposed in the second recessed portion for receiving reflective light from the human skin through the second light transmissive sheet.

7. The wrist-worn type electronic device claim 6, wherein the optical transceiver set further comprises a second light emitting diode unit, the second light emitting diode unit is disposed in the first recessed portion for emitting lights to the human skin through the first light transmissive sheet, wherein the first recessed portion surrounds the second recessed portion.

8. The wrist-worn type electronic device claim 1, wherein the signal indicating module comprises:
a watch face image disposed on one surface of the device body;
a plurality of indicating pointers rotatably disposed on the watch face image, the indicating pointers indicate at least one physiological information; and
a plurality of driving motors disposed in the device body, electrically connected to the control unit, wherein each of the driving motors individually connects to control one of the indicating pointers.

9. The wrist-worn type electronic device of claim 1, further comprising:
a manual switch disposed on the device body, electrically connected to the control unit, for being manually operated to enable and disable the wireless communication module to electrically connect to the external device.

10. A wrist-worn type electronic device, comprising:
a device body comprising a first recessed portion, a second recessed portion, a light shielding portion, a first light transmissive sheet and a second light transmissive sheet, the light shielding portion is arranged between the first recessed portion and the second recessed portion, the first light transmissive sheet is totally received within the first recessed portion, and the second light transmissive sheet is totally received within the second recessed portion;
a physiological information induction module comprising:
a circuit board disposed in the device body;
a first light emitting diode unit mounted on the circuit board, and disposed in the first recessed portion for emitting lights to the human skin through the first light transmissive sheet; and
a photodiode unit mounted on the circuit board, and disposed in the first recessed portion for receiving reflective lights from the human skin through the second light transmissive sheet;
a signal indicating module disposed on the device body;
a wireless communication module for wirelessly and electrically connected to an external device; and
a control unit electrically connected to the photodiode unit, the first light emitting diode unit, the wireless communication module and the signal indicating module.

11. The wrist-worn type electronic device claim 10, wherein the physiological information induction module further comprises a second light emitting diode unit, the second light emitting diode unit is disposed in the first recessed portion, electrically connected to the control unit, wherein the second recessed portion surrounds the first recessed portion.

12. The wrist-worn type electronic device of claim 10, wherein the physiological information induction module further comprises a first electrocardiogram electrode and a second electrocardiogram electrode which are oppositely arranged on the device body, and both electrically connected to the control unit, wherein the first electrocardiogram electrode is an annular conductive frame surrounding the signal indicating module.

13. The wrist-worn type electronic device claim 10, wherein the signal indicating module comprises:
a watch face image disposed on one surface of the device body;
a plurality of indicating pointers rotatably disposed on the watch face image, the indicating pointers indicate at least one physiological information; and
a plurality of driving motors disposed in the device body, electrically connected to the control unit, wherein each of the driving motors individually connects to control one of the indicating pointers.

14. The wrist-worn type electronic device of claim 10, further comprising:
a manual switch disposed on the device body, electrically connected to the control unit, for being manually operated to enable and disable the wireless communication module to electrically connect to the external device.
